(19) United States
(12) Patent Application Publication
(10) Pub. No.: US 2012/0289565 A1
(43) Pub. Date: Nov. 15, 2012

(54) COMBINATIONS OF TOLTERODINE AND SALIVARY STIMULANTS FOR THE TREATMENT OF OVERACTIVE BLADDER

(75) Inventors: Mehdi PABORJI, Cupertino, CA (US); Kenneth L. DUCHIN, Delray Beach, FL (US); Wendy Jade Limayo HERNANDEZ, Redwood City, CA (US); Roger S. FLUGEL, Redwood City, CA (US)

(73) Assignee: TheraVida, Inc., Mountain View, CA (US)

(21) Appl. No.: 13/476,870

(22) Filed: May 21, 2012

Related U.S. Application Data
(63) Continuation of application No. 13/467,177, filed on May 9, 2012.
(60) Provisional application No. 61/484,655, filed on May 10, 2011.

(51) Int. Cl. A61K 31/4178; A61P 13/00

(52) U.S. Cl. 514/397

(57) ABSTRACT

Disclosed herein are pharmaceutical compositions comprising a therapeutically effective amount of extended release tolterodine, or a pharmaceutically acceptable salt thereof, and a therapeutically effective amount of pilocarpine, or a pharmaceutically acceptable salt thereof. Also disclosed herein are methods of treating a patient suffering from overactive bladder, the method comprising identifying a patient in need thereof, and administering to the patient a therapeutically effective amount of extended release tolterodine, or a pharmaceutically acceptable salt thereof, and a therapeutically effective amount of pilocarpine, or a pharmaceutically acceptable salt thereof. Also disclosed herein are methods of alleviating a side effect of treatment for overactive bladder in a patient suffering therefrom, the method comprising identifying a patient in need thereof, and administering to the patient a therapeutically effective amount of extended release tolterodine, or a pharmaceutically acceptable salt thereof, and a therapeutically effective amount of pilocarpine, or a pharmaceutically acceptable salt thereof.

### RELATED APPLICATIONS

This application is a continuation of U.S. application Ser. No. 13/467,177, filed May 9, 2012, by Mehdi Paborji et al., and entitled “COMBINATIONS OF TOLTERODINE AND SALIVARY STIMULANTS FOR THE TREATMENT OF OVERACTIVE BLADDER,” which in turn claims priority to U.S. Provisional Application No. 61/484,655, filed May 10, 2011, by Mehdi Paborji et al., and entitled “COMBINATIONS OF TOLTERODINE AND SALIVARY STIMULANTS FOR THE TREATMENT OF OVERACTIVE BLADDER,” which is incorporated herein by reference in its entirety.

### FIELD OF THE INVENTION

The present invention is in the field of pharmaceutical compositions and methods of using the same for the treatment of overactive bladder and reduction of various side effects thereof.

### BACKGROUND OF THE INVENTION

Overactive bladder (OAB) is characterized by involuntary contractions of the detrusor muscle during bladder filling. These contractions may be asymptomatic or may cause the three common symptoms that clinically define OAB: frequency of urination; urgency; and urge, or reflex incontinence. Frequency is an increase in the number of micturitions, to as many as eight or more a day. Urgency is the strong and sudden desire to urinate. Urge incontinence, or reflex incontinence, is the situation where the urge to urinate cannot be controlled and the patient wets his/her clothing. Nocturia, or nighttime urinary frequency that disturbs sleep (more than twice a night), is often included as a fourth symptom. The symptoms of OAB may appear individually or together, and it is not known whether they have a pathologic or neurogenic cause.

Several classes of medications have been used to treat and manage OAB, including antimuscarinic agents. Antimuscarinic agents, which exert their effects at muscarinic receptors and suppress or diminish the intensity of involuntary detrusor muscle contractions, are the first-choice pharmacotherapy for OAB, and may be the only therapy available whose efficacy is not in question. Tolterodine tartrate is one of the most extensively studied of the antimuscarinic agents, and one of the most widely used. A major limitation of the use of tolterodine is that it lacks specificity for bladder tissue, with resultant bothersome side effects, such as dry mouth, constipation, effects on cognition, impaired sleep, etc.

The use of tolterodine tartrate in combination with pilocarpine has been previously disclosed, for example in U.S. Pat. Nos. 7,666,894, 7,678,821, 7,781,472.

### SUMMARY OF THE INVENTION

Disclosed herein are pharmaceutical compositions comprising a therapeutically effective amount of extended release tolterodine, or a pharmaceutically acceptable salt thereof, and a therapeutically effective amount of a muscarinic agonist. Also disclosed herein are methods of treating a patient suffering from overactive bladder, the method comprising identifying a patient in need thereof, and administering to the patient a therapeutically effective amount of extended release tolterodine (e.g. Detrol® LA), or a pharmaceutically acceptable salt thereof, and a therapeutically effective amount of a muscarinic agonist. Also disclosed herein are methods of alleviating a side effect of treatment for overactive bladder in a patient suffering therefrom, the method comprising identifying a patient in need thereof, and administering to the patient a therapeutically effective amount of extended release tolterodine, or a pharmaceutically acceptable salt thereof, and a therapeutically effective amount of a muscarinic agonist.

### BRIEF DESCRIPTION OF THE DRAWINGS

FIG. 1 is a graph showing the results of a clinical study on saliva formation when the subject was given a) no drug (); b) 4 mg Detrol® LA (▴); and c) 4 mg Detrol® LA followed by 2.5 mg of pilocarpine 2.5 hours after the administration of tolterodine (▪).

FIG. 2 is a graph showing the results of a clinical study on saliva formation when the subject was given a) no drug (); b) 4 mg Detrol® LA (▴);and c) 4 mg Detrol® LA followed by 5 mg of pilocarpine 4 hours after the administration of tolterodine (▪).

FIGS. 3A and 3B, which show the results of two separate runs of the same experiment conducted 2 weeks apart, are graphs showing the results of a clinical study on saliva formation when the subject was given a) no drug (); b) 4 mg Detrol® LA (▴);and c) 4 mg Detrol® LA followed by 5 mg of pilocarpine 2.5 hours after the administration of tolterodine (♦ in FIG. 3A, □ in FIG. 3B).

### DETAILED DESCRIPTION OF THE PREFERRED EMBODIMENTS

The major limitations of treatment of overactive bladder (OAB) are the dry mouth and constipation side effects. The current approach to address the dry mouth is development of sustained release of the active moiety, such as tolterodine in the form of Detrol® LA, which is disclosed in U.S. Pat. No. 6,911,217, incorporated by reference herein in its entirety. However, patients taking the longer-acting sustained release formulation of tolterodine still suffer from these side effects and thus their quality of life is hampered significantly to the extent that the majority of patients discontinue the mediations after about 4-6 months.

The inventor of the present application had previously discovered that combining the immediate release form of tolterodine with the immediate release form of pilocarpine, a muscarinic agonist that increases salivation, can overcome the dry mouth side effect and increase patient comfort and quality of life. See, for example, U.S. Pat. No. 7,678,821 and U.S. Patent Application Publication 2010/0152263, both of which are incorporated by reference herein in their entirety. The present disclosure is focused on combinations with the sustained release form of tolterodine.

Thus, in the first aspect, the present invention relates to a pharmaceutical composition comprising a therapeutically effective amount of a sustained release formulation of tolterodine, or a pharmaceutically acceptable salt thereof, and a therapeutically effective amount of a muscarinic agonist.

Tolterodine, which has the chemical name (R)-2-[3-[bis(1-methylethyl-amino]-1-phenylpropyl]-4-methylphenol [R—(R*,R*)]-2,3-dihydroxybutanedionic acid, is a muscarinic receptor antagonist and is the active ingredient found in the product Detrol® LA (as tolterodine tartrate).

Within the context of the present disclosure, a “muscarinic agonist” is a compound that modulates, i.e., agonizes, the activity of a muscarinic receptor either directly or indirectly. A muscarinic agonist acts directly on the muscarinic receptors when the muscarinic agonist itself binds to the muscarinic receptor and modulates its activity. A muscarinic agonist acts indirectly on the muscarinic receptors when the muscarinic agonist stimulates the production of an endogenous muscarinic agonist, which in turn agonizes the muscarinic receptors. An endogenous muscarinic agonist is a natural binding partner of the muscarinic receptors and is produced by the body of the subject itself. An example of an endogenous muscarinic agonist is acetylcholine.

In certain embodiments, the muscarinic agonists selected from the group consisting of pilocarpine, cevimeline, anethole trithione, aclatonium napadisilate, and yohimbine, or a pharmaceutically acceptable salt or prodrug thereof. In further embodiments, the muscarinic agonist is pilocarpine, or a pharmaceutically acceptable salt or prodrug thereof. In other embodiments, the second compound is cevimeline, or a pharmaceutically acceptable salt or prodrug thereof.

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, succinic acid, tartaric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl) methylamine, and salts thereof with amino acids such as arginine, lysine, and the like.

Throughout the present disclosure, when a particular compound is named, it is understood that the name refers to both the free base, or free acid, of the compound, and the pharmaceutically acceptable salts thereof. Thus, for example, the scope of the term “tolterodine” covers both the free base of tolterodine, i.e., (R)-2-[3-[bis(1-methylethyl-amino]-1-phenylpropyl]-4-methylphenol [R—(R*,R*)]-2,3-dihydroxybutanedionic acid, and its various pharmaceutically acceptable salts, for example tolterodine tartrate.

The compounds useful for the compositions and methods described herein may be used in various formulations. Certain formulations affect the rate at which the compound enters the blood stream of the patient. Thus, some formulations are immediate release formulations while other formulations are delayed release, sustained release, or extended release formulations.

Thus, in some embodiments, disclosed herein are combinations where tolterodine, or a pharmaceutically acceptable salt thereof, is in an extended release formulation, while the muscarinic agonist is in an immediate release formulation. In other embodiments, both tolterodine, or a pharmaceutically acceptable salt thereof, and the muscarinic agonist are in an extended release formulation.

By “extended release formulation” of tolterodine it is meant a formulation of tolterodine, similar to that found in Detrol® LA, where tolterodine is administered once a day.

The compositions described herein are particularly useful in alleviating the major side effects in the treatment of OAB, namely dry mouth, discomfort around the mouth, difficulty speaking secondary to dry mouth, degree of difficulty chewing food secondary to dry mouth, and/or lack of quality of sleep, improving tolerability, and enhancing patient compliance while increasing the patient's quality of life.

In another aspect, the present invention relates to a method of treating a patient comprising administering to a patient in need thereof a therapeutically effective amount of a sustained release formulation of tolterodine, or a pharmaceutically acceptable salt thereof, and a therapeutically effective amount of a muscarinic agonist.

A patient in need of the treatment methods disclosed herein may be a patient who suffers from overactive bladder. The patient may also be one who finds current therapies for overactive bladder uncomfortable and/or the side effects of the therapy, such as the dry mouth, intolerable enough so as to require adjunct therapy to alleviate the side effects. The patient may also be one who is considering discontinuing therapy for overactive bladder due to the side effects of the therapy. In some embodiments, a patient who is recently diagnosed with overactive bladder but yet has not been treated therefor is a patient in need of the treatment methods and compositions disclosed herein. In these embodiments, the patient begins the therapy using the methods and combinations disclosed herein so that the patient does not experience any of the side effects, or experience the side effects to a lesser degree.

In some embodiments tolterodine, or a pharmaceutically acceptable salt thereof, and the muscarinic agonist are administered more or less simultaneously. In other embodiments tolterodine, or a pharmaceutically acceptable salt thereof, is administered prior to the muscarinic agonist. In yet other embodiments, tolterodine, or a pharmaceutically acceptable salt thereof, is administered subsequent to the muscarinic agonist.

It should be noted that simply taking commercially available the muscarinic agonist, e.g., pilocarpine HCl, e.g., Salagen® tablets, or any other salivary gland stimulants in conjunction with an OAB drug is not effective to alleviate the dry mouth side effect. The disclosed methods of therapy and therapeutic combinations are directed to matching the pharmacokinetic profile of the muscarinic agonist with the pharmacokinetic profiles of tolterodine, or a pharmaceutically acceptable salt thereof.

The present inventors have surprisingly discovered that if the extended release formulation of tolterodine, or a pharmaceutically acceptable salt thereof, and the muscarinic agonist are administered such that the peak plasma concentration for tolterodine occurs at nearly the same time after administration as the peak plasma concentration for the muscarinic agonist, then the patient will not receive the most efficacious combination of the two compounds. That is, in this situation, the patient still suffers from dry mouth and the related side effects that would render the patient uncomfortable. Instead, if the two compounds are administered such that the peak plasma concentration for the muscarinic agonist occurs at a time before the peak plasma concentration for tolterodine, then the patient receives the maximum therapeutic effect of the combination.

Similarly, if the extended release formulation of tolterodine, or a pharmaceutically acceptable salt thereof, and the muscarinic agonist are administered such that the time point at which the lowest saliva flow occurs because of the action of tolterodine nearly corresponds to the time point at which the highest saliva flow occurs because of the action of the muscarinic agonist, then the patient will not receive the most efficacious combination of the two compounds. Instead, if the two compounds are administered such that the time point at which the lowest saliva flow occurs because of the action of tolterodine after the time point at which the highest saliva flow would have occurred because of the action of the muscarinic agonist in the absence of tolterodine, then the patient receives the maximum therapeutic effect of the combination.

In some embodiments in the above methods, tolterodine, or a pharmaceutically acceptable salt thereof, and the muscarinic agonist are administered such that the ratio of their plasma concentrations, at a given point in time following their administration, is a predetermined value. Those of ordinary skill in the art recognize that the ratio of plasma concentrations is not necessarily the same as the ratio of the amount of compound administered. Compounds are digested differently in the gut, pass the gut wall differently, and have a different rate of first-pass metabolism in the liver. Furthermore, the clearance rate by the kidney is different for various compounds. Thus, for example, even if two compounds are administered in equivalent molar amounts, their plasma concentrations at a point in time after the administration may be significantly different. The methods disclosed herein take into account the pharmacokinetics of drug intake and metabolism, such that the ratio of the two compounds at the time of administration is adjusted so that the two compounds would have a predetermined concentration ratio in the plasma.

Thus, the two compounds may be administered simultaneously, but be formulated such that the delay in their release causes maximum therapeutic effect for the patient. In some of the embodiments when the two compounds are administered simultaneously, the two compounds are within one dosage form.

In some embodiments the dosage form is designed as sustained release of one agent combined with either sustained release or immediate release of the second agent to ensure maximum therapeutic effect. Further the dosage from can be designed based on the pharmacokinetic profiles where the peak plasma concentration of one compound, for example the muscarinic agonist, corresponds to maximum amount of mouth dryness caused by tolterodine.

Thus, in another aspect, the present invention relates to a method of increasing intrinsic bladder capacity, comprising administering to a patient in need thereof a therapeutically effective amount of tolterodine, or a pharmaceutically acceptable salt thereof, and a therapeutically effective amount of a muscarinic agonist.

The term “pharmaceutical composition” refers to a mixture of a compound of the invention with other chemical components, such as diluents, lubricants, bulking agents, desentegrant or carriers. The pharmaceutical composition facilitates administration, for example orally, of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

In certain embodiments, the same substance can act as a carrier, diluent, or excipient, or have any of the two roles, or have all three roles. Thus, a single additive to the pharmaceutical composition can have multiple functions.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen and desired pharmacokinetic profiles of each component of combination therapy. Any of the well-known techniques, carriers, and excipients may be used as suitable and as understood in the art; e.g., in Remington's Pharmaceutical Sciences, above.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

Many of the compounds used in the pharmaceutical combinations of the invention may be provided as salts with pharmaceutically compatible counterions. Pharmaceutically compatible salts may be formed with many acids, including but not limited to hydrochloric, sulfuric, acetic, lactic, tartaric, malic, succinic, and the like. Salts tend to be more soluble in aqueous or other protonic solvents than are the corresponding free acids or base forms.

Typically, the dose range of the composition administered to the patient can be from about 0.010 to 1000 mg/kg of the patient's body weight. The dosage may be a single one or a series of two or more given in the course of one or more days, as is needed by the patient. Note that for tolterodine and the muscarinic agonist, human dosages for treatment of at least some condition have been established. For example, for tolterodine the preferred dosage is between 0.1 mg to 50 mg, and the more preferred dosage is between 1 mg to 30 mg. Other dose ranges include between 1 to 10 mg, between 2 mg to 8 mg, between 3 to 6 mg, between 4 mg to 5 mg. The dose may also be at 1 mg, 2 mg, 3 mg, 4 mg, 5 mg, 6 mg, 7 mg, 8 mg, or 10 mg. For pilocarpine, the preferred dosage is between 0.1 mg to 50 mg, and the more preferred dosage is between 1 mg to 30 mg. Other dose ranges include between 2 to 20 mg, between 3 to 25 mg, and between 4 to 20 mg. The dose may also be at 1 mg, 2 mg, 3 mg, 4 mg, or 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 13 mg, and 15 mg.

Although the exact dosage can be determined on a drug-by-drug basis, in most cases, some generalizations regarding the dosage can be made. The daily dosage regimen for an adult human patient may be, for example, an oral dose of between 0.001 mg and 1000 mg of each ingredient, preferably between 0.01 mg and 500 mg, for example 1 to 200 mg or each ingredient of the pharmaceutical compositions of the present invention or a pharmaceutically acceptable salt thereof calculated as the free base or free acid, the composition being administered 1 to 4 times per day or per week. Alternatively the compositions of the invention may be administered by continuous delivery such as sustained, delayed, or extended release, preferably at a dose of each ingredient up to 500 mg per day. Thus, the total daily dosage by oral administration of each ingredient will typically be in the range 0.1 mg to 2000 mg. Suitably the compounds will be administered for a period of continuous therapy, for example for a day, a week or more, or for months or years.

The amount of composition administered will, of course, be dependent on the subject being treated, on the subject's weight, the severity of the affliction, the extent of deficiency in a cytochrome P450 2D6 enzyme, the manner of administration and the judgment of the prescribing physician.

It will be understood by those of skill in the art that numerous and various modifications can be made without departing from the spirit of the present invention. Therefore, it should be clearly understood that the forms of the present invention are illustrative only and are not intended to limit the scope of the present invention.

### EXAMPLES

The examples below are non-limiting and are merely representative of various aspects of the invention.

### Example 1

### Case Study for a Combination of Tolterodine and Pilocarpine

In this study, the effect of tolterodine (Detrol® LA), pilocarpine, the combination of the two, and placebo was measured in separate, yet identical, studies in a single individual.

### Study Protocol:

The following protocol was for a pilot human study to evaluate the extent of saliva flow rate following the administration of 4 mg Detrol® LA capsule and 5 mg pilocarpine HCl tablet after 2.5 hrs from tolterodine administration. The subject was a healthy, white, male, 53 years old. The subject performed the following:
1. Fast the night before.
2. No coffee or soda 8 hrs prior to taking the first dose.
3. Record time voiding and measure urine if possible.
4. Record extent of dryness (Very dry, dry, and not dry).
5. Record any adverse event.
6. Chew a 1″×1″ piece of Parafilm (American National Can (Neenah, Wis. 54956) Bar code No. 7-466676999) over 2 minutes.
7. Collect wetted Parafilm and saliva into a tared container (pre-weighed container).
8. Measure the amount of saliva collected.
9. Plot Saliva collected per 2 min time intervals against time.

The subject fasted overnight, but had 240 mL of water 1 hr prior to dose. The water continued ad lib until 1 hr pre and 1 hr post dose, other than 120 mL with Detrol® LA and 120 mL with 5 mg Salagen tablet. For lunch, the subject consumed a light sandwich. No coffee or soda or alcohol beverages was consumed.

Table 1 shows a summary of study results for a dose-response and time course of pilocarpine reversal of dry mouth.

TABLE 1

| | | | Time | |
|---|---|---|---|---|
| | | | Pilocarpine | |
| | Detrol ® | | Administered | |
| | LA | Dose of | Relative to | |
| Run | Dose | Pilocarpine | Detrol ® LA | Observations |
| 1 | 4 mg | 0 | 0 | Active control - |
| | | | | reduced salivation |
| | | | | from 3 to 9 hrs. |
| 2 | 4 mg | 5 mg | 4 hrs | Still significant |
| | | | | decrease in saliva |
| | | | | production (dry |
| | | | | mouth) from 3 to |
| | | | | about 6 hrs. |
| 3 | 4 mg | 5 mg | 2.5 hrs | Good blunting of dry |
| | | | | mouth, decrease in |
| | | | | saliva production |
| | | | | caused by Detrol ® |
| | | | | LA was effectively |
| | | | | counteracted. |
| | | | | Corrected baseline |
| | | | | saliva values were in |
| | | | | the positive portion of |
| | | | | the saliva vs. time |
| | | | | plot. |
| 4 | 4 mg | 2.5 mg | 2.5 hrs | Nearly the same nadir |
| | | | | as Detrol ® LA alone, |
| | | | | but the duration of dry |
| | | | | mouth was not as long |
| | | | | as Detrol ® LA. |
| 5 | 4 mg | 5 mg | 2.5 hrs | Repeat of run#3 |
| | | | | (2 weeks later) - good |
| | | | | blunting of dry mouth, |
| | | | | decrease in saliva |
| | | | | production caused by |
| | | | | Detrol ® LA was |
| | | | | effectively |
| | | | | counteracted and was |
| | | | | found to be |
| | | | | reproducible |
| | | | | compared to run#3. |
| 6 | 0 | 0 | 0 | Control with no drug - |
| | | | | positive saliva |
| | | | | production was |
| | | | | observed over the |
| | | | | entire period of 12 hrs. |

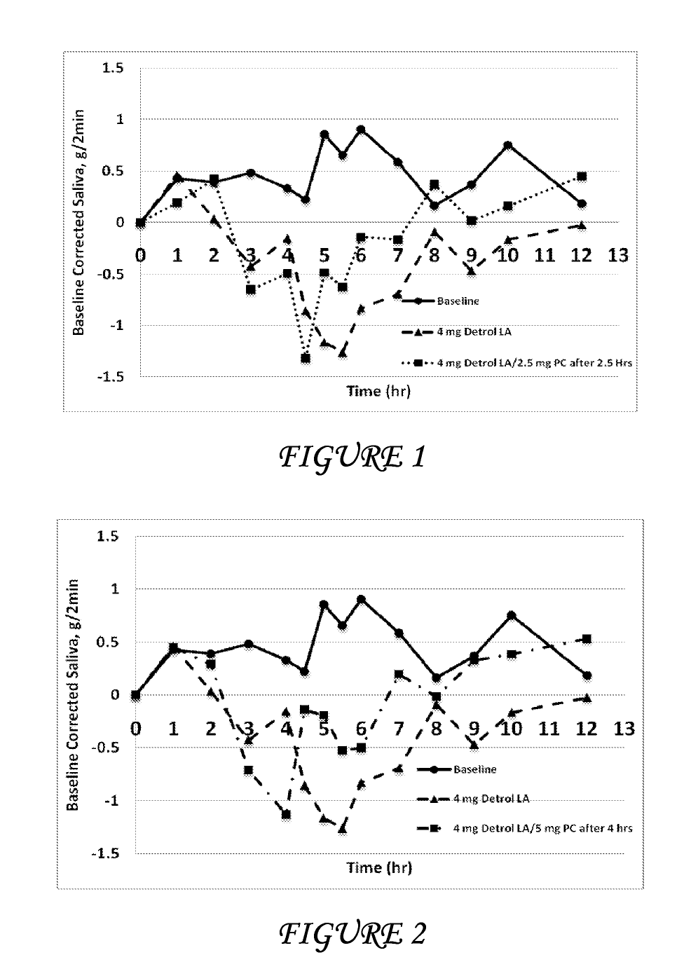

FIG. 1 shows three separate lines. The first () is the baseline corrected saliva output for the subject during the course of 13 hours. The subject in this case did not take any medications, but followed the study protocol outlined above with respect to food and fluid intake. As the graph shows, there is a natural variation of less than about ±0.5 g/2 minutes of saliva during the course of the study. The second line (▴) is the corrected saliva output for the subject having taken 4 mg Detrol® LA during the course of 13 hours. The graph shows the extent of saliva output depression caused by tolterodine. The maximum dry mouth occurs at about 6 hours after the administration of tolterodine. The third line (▪) shows the corrected saliva output for the subject having taken 4 mg Detrol® LA followed by 2.5 mg of pilocarpine at 2.5 hours after the administration of tolterodine. As can be seen, the maximum saliva output depression occurs earlier than when no pilocarpine was administered. However, saliva output is depressed to the same extent as when no pilocarpine was administered.

FIG. 2 also shows three separate lines. The first two lines ( and ▴) are identical to the respective lines of FIG. 1. The third line (▪) shows the corrected saliva output for the subject having taken 4 mg Detrol® LA followed by 5 mg of pilocarpine at 4 hours after the administration of tolterodine. As can be seen, the maximum saliva output depression occurs earlier than when no pilocarpine was administered. However, saliva output is depressed to the same extent as when no pilocarpine was administered suggesting that administering pilocarpine at about 4 to 6 hours close to the minimal point of salivary output depression does not lead to improved tolerability or bringing salivary flow output to normal level or baseline level.

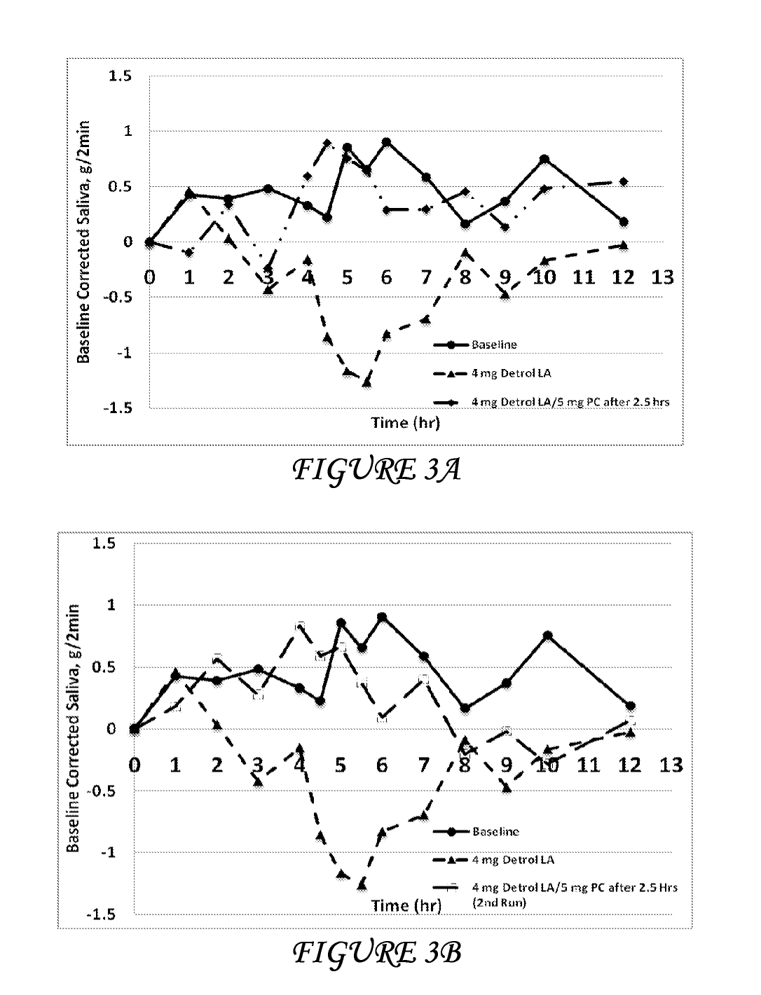

FIGS. 3A and 3B, which show the results of two separate runs of the same experiment, also show three separate lines. The first two lines ( and ▴) are identical to the respective lines of FIGS. 1 and 2. The third line (♦ in FIG. 3A, □ in FIG. 3B) shows the corrected saliva output for the subject having taken 4 mg Detrol® LA followed by 5 mg of pilocarpine at 2.5 hours after the administration of tolterodine. As can be seen, the saliva output is similar to that of the baseline, i.e., when the subject received no drug at all. Therefore, administration of 5 mg of pilocarpine 2.5 hours after the administration of tolterodine restored normal saliva output for the subject for the course of study. Note that in this case, pilocarpine is administered at about 3 hours earlier than the point where the salivary depression caused by tolterodine would have been maximal.

The data in the FIGS. 1-3 are shown in tabular format below. Table 2 lists the absolute values of the weight of the collected saliva (g/2 min) at the various time points for the different study arms.

TABLE 2

| | | | 4D-5P- | 4D-5P- | | | |
|---|---|---|---|---|---|---|---|
| Time, | | 4D- | 2.5^{c} | 2.5^{c} | | 4D-2.5P- | Base- |
| Hrs | 4D^{a} | 5P-4^{b} | (1^{st} run) | (2^{nd} run) | Avg.^{d} | 2.5^{e} | line |
| 0 | 2.94 | 2.386 | 2.59 | 2.769 | 2.6795 | 2.726 | 2.645 |
| 1 | 3.4 | 2.835 | 2.495 | 2.95 | 2.7225 | 2.919 | 3.071 |
| 2 | 2.975 | 2.675 | 2.925 | 3.335 | 3.13 | 3.145 | 3.035 |
| 3 | 2.515 | 1.675 | 2.355 | 3.047 | 2.701 | 2.074 | 3.125 |
| 4 | 2.785 | 1.254 | 3.184 | 3.6 | 3.392 | 2.235 | 2.975 |
| 4.5 | 2.085 | 2.245 | 3.485 | 3.358 | 3.4215 | 1.41 | 2.867 |
| 5 | 1.775 | 2.191 | 3.345 | 3.43 | 3.3875 | 2.24 | 3.5 |
| 5.5 | 1.68 | 1.86 | 3.235 | 3.145 | 3.19 | 2.1 | 3.3 |
| 6 | 2.11 | 1.885 | 2.88 | 2.861 | 2.8705 | 2.585 | 3.55 |
| 7 | 2.245 | 2.58 | 2.885 | 3.167 | 3.026 | 2.56 | 3.23 |
| 8 | 2.85 | 2.37 | 3.045 | 2.565 | 2.805 | 3.096 | 2.809 |
| 9 | 2.47 | 2.71 | 2.725 | 2.75 | 2.7375 | 2.745 | 3.015 |
| 10 | 2.775 | 2.77 | 3.07 | 2.484 | 2.777 | 2.886 | 3.399 |
| 12 | 2.915 | 2.915 | 3.135 | 2.835 | 2.985 | 3.177 | 2.829 |

^{a}4D = 4 mg Detrol ® LA
^{b}4D-5P-4 = 4 mg Detrol ® LA followed by 5 mg pilocarpine 4 hrs after Detrol ® LA.
^{c}4D-5P-2.5 = 4 mg Detrol ® LA followed by 5 mg pilocarpine 2.5 hrs after Detrol ® LA.
^{d}Avg. is the average of the two runs of 4D-5P-2.5.
^{e}4D-2.5P-2.5 = 4 mg Detrol ® LA followed by 2.5 mg pilocarpine 2.5 hrs after Detrol ® LA.

Table 3 lists the baseline corrected values shown in Table 2. To obtain the values in Table 3, the value for time=0 hours in Table 2 is subtracted from the values for the other time points in each column.

TABLE 3

| Time, | | | 4D-5P-2.5 | 4D-5P-2.5 | | Base- |
|---|---|---|---|---|---|---|
| Hrs | 4D | 4D-5P-4 | (1^{st} run) | (2^{nd} run) | 4D-2.5P-2.5 | line |
| 0 | 0 | 0 | 0 | 0 | 0 | 0 |
| 1 | 0.46 | 0.449 | −0.095 | 0.193 | 0.181 | 0.426 |
| 2 | 0.035 | 0.289 | 0.335 | 0.419 | 0.566 | 0.39 |
| 3 | −0.425 | −0.711 | −0.235 | −0.652 | 0.278 | 0.48 |
| 4 | −0.155 | −1.132 | 0.594 | −0.491 | 0.831 | 0.33 |
| 4.5 | −0.855 | −0.141 | 0.895 | −1.316 | 0.589 | 0.222 |
| 5 | −1.165 | −0.195 | 0.755 | −0.486 | 0.661 | 0.855 |
| 5.5 | −1.26 | −0.526 | 0.645 | −0.626 | 0.376 | 0.655 |
| 6 | −0.83 | −0.501 | 0.29 | −0.141 | 0.092 | 0.905 |
| 7 | −0.695 | 0.194 | 0.295 | −0.166 | 0.398 | 0.585 |
| 8 | −0.09 | −0.016 | 0.455 | 0.37 | −0.204 | 0.164 |
| 9 | −0.47 | 0.324 | 0.135 | 0.019 | −0.019 | 0.37 |
| 10 | −0.165 | 0.384 | 0.48 | 0.16 | −0.285 | 0.754 |
| 12 | −0.025 | 0.529 | 0.545 | 0.451 | 0.066 | 0.184 |

As can be seen from the data, the maximum saliva depression point for Detrol® LA occurs at about 5.5 hours after its administration. Previous studies, for example FIG. 1 of U.S. Pat. No. 7,678,821, which figure and the related discussion in the specification are incorporated by reference herein, have shown that maximum saliva output due to 5 mg of pilocarpine occurs about 30 minutes after its administration. One would expect for pilocarpine to retard the saliva depression of Detrol® LA most effectively, pilocarpine would need to be administered about 30 minutes before the maximum depression point due to Detrol® LA. That is, one would expect that pilocarpine would need to be administered about 5 hours after the administration of Detrol® LA. However, the results presented herein show unexpectedly that best results are obtained when pilocarpine is administered at about 2.5 hours after the administration of Detrol® LA.

### Example 2

### Clinical Study Protocol Synopsis

A study was conducted to evaluate the effect of tolterodine (Detrol® LA) and pilocarpine in overactive bladder patients. The objectives of the study were to determine degree of dry mouth after oral administration of tolterodine and pilocarpine, and to determine the effect of the combination on number of voids, and number of incontinence episodes.

Subjects who have reasonable control of OAB symptoms (urinary frequency ≦13 voids/day and ≦1 incontinence episode/day) and have good tolerability (excluding dry mouth) while taking a stable dose of Detrol® LA (4 mg/day) were recruited to participate in this evaluation. All subjects were administered Detrol® LA for at least 4 to 6 weeks before being administered the combination therapy. The subjects were asked to record their OAB symptoms and status of dry mouth symptoms in a 3-day diary.

The subjects were asked to take pilocarpine (5 mg) at 2.5 hours after Detrol® LA is taken. The combination was given for at least 2 weeks. At the end of the 2-week period, a 3-day diary for voiding function, incontinence episodes, and dry mouth evaluation was collected.

Data related to voiding information were collected in diaries that were recorded over 3 consecutive days at the end of each treatment period. Self-assessments of dry mouth and other related activities/functions were made using validated 100 mm visual analog scales (VAS) that were completed on each of the three diary days. On the VAS, the value of 0 mm meant that there was no adverse symptom, whereas the value of 100 mm meant that the adverse symptom was at a highly intolerable level. The average value obtained over the 3 days was used as the value for the treatment period whether it was baseline (Detrol® LA alone) or the study periods (combination of Detrol® LA and pilocarpine).

The mean value (±standard deviation (SD)) for each 3-day measurement for each patient was calculated. The data point before the commencement of Period 1 was considered to be baseline. Baseline correction was applied by subtracting the baseline value from the data point at the end of the 2-week Period. The baseline corrected values are shown in the tables below.

Table 4 shows the baseline corrected value for the number of voids (micturitions) per day and the number of incontinence episodes (IE) per day. As can be seen, the addition of pilocarpine to Detrol® LA does not adversely affect the efficacy of Detrol® LA, because the number of micturitions and IEs do not worsen after the introduction of pilocarpine. Therefore, the addition of a muscarinic agonist to the muscarinic antagonist therapy does not alter the mechanism of action of the antagonist.

More significantly, the dry mouth and the related adverse symptoms decrease significantly. Table 5 shows the baseline corrected VAS values for dry mouth. As can be seen, at the end of the 2-week treatment, the VAS value decreases significantly. The data clearly show that pilocarpine can effectively negate the adverse dry mouth effect of Detrol® LA in this study.

TABLE 4
Variation of Change from Baseline in Micturition and Incontinence
Episodes of Tolterodine (Detrol ® LA) with Treatment

| | Change from Baseline | | Change from Baseline in |
|---|---|---|---|
| | in Micturition Per Day* | | Incontinence Episodes |
| Subject No. | Baseline | 2-week Treatment | 2-week Treatment |
| 001 | 0.0 | −3.0 | 0.7 |
| 002 | 0.0 | 0.7 | 0.0 |
| 003 | 0.0 | 1.3 | 0.3 |
| 004 | 0.0 | 0.7 | 0.0 |
| Mean | 0.0 | −0.1 | 0.3 |
| SD | 0.0 | 2.0 | 0.3 |

*Each value represents mean of micturition value per day collected over a 3-consecutive day period.

TABLE 5
Variation of Change from Baseline in VAS Values of Dry Mouth of
Treatment with Tolterodine (Detrol ® LA)

Change from Baseline
in VAS*

| Subject No. | Baseline | 2-week Treatment |
|---|---|---|
| 001 | 0.0 | 0.7 |
| 002 | 0.0 | −7.0 |
| 003 | 0.0 | −50.0 |
| 004 | 0.0 | −40.0 |
| Mean | 0.0 | −24.1 |
| SD | 0.0 | 24.7 |

*Each value represents mean of VAS scores of dry mouth collected over a 3-consecutive day period.

Table 6 shows the VAS values for other, secondary adverse symptoms related to dry mouth. These include the general feeling in the mouth, quality of sleep, ease of speaking, and ease of swallowing. As the data show, all of these metrics also improved in a sustained and consistent way when pilocarpine was added to Detrol® LA-therapy.

TABLE 6
Variation of Change from Baseline in VAS Values of other Dry Mouth
Related Adverse Symptoms of Treatment with Tolterodine (Detrol ® LA)

| | | Feeling in | | | |
|---|---|---|---|---|---|
| | | Mouth | Sleeping | Speaking | Swallowing |
| Subject | | 2-week | 2-week | 2-week | 2-week |
| No. | Baseline | Treatment | Treatment | Treatment | Treatment |
| 001 | 0.0 | −8.0 | −21.3 | −27.0 | −20.3 |
| 002 | 0.0 | −0.7 | −27.3 | −20.0 | −13.7 |
| 003 | 0.0 | −39.7 | −1.3 | −6.7 | −1.7 |
| 004 | 0.0 | −14.0 | −25.0 | −31.3 | −35.0 |
| Mean | 0.0 | −15.6 | −18.8 | −21.3 | −17.7 |
| SD | | 17.0 | 11.9 | 10.8 | 13.9 |

*Each value represents mean of VAS scores collected over a 3-consecutive day period

### Example 3

### Clinical Study Protocol Synopsis

A study is conducted to evaluate the effect of single doses of tolterodine (Detrol® LA) and pilocarpine, alone and in combination versus placebo on salivary output in healthy volunteers. The objectives of the study are to determine salivary flow and degree of dry mouth after oral administration of tolterodine and pilocarpine, alone and in combination, vs. placebo, and to determine the effect of tolterodine and pilocarpine, alone and in combination, on urine volume/void and vital signs.

At each treatment period, following an overnight fast, subjects enter the clinic and after baseline measurements have been made, they are randomized to one of the following groups:
Tolterodine (Detrol® LA, 4 mg) followed 2.5 hours later by placebo
Pilocarpine (5 mg) followed 2.5 hours later by placebo
Placebo followed 2.5 hours later by placebo
Tolterodine (Detrol® LA, 4 mg) followed 2.5 hours by pilocarpine (5 mg)
Tolterodine (Detrol® LA, 4 mg) followed 4 hours by pilocarpine (5 mg)

The following measurements are made just prior to and at frequent intervals for up to 12 hours post dose:
Salivary flow is determined by chewing a piece of Parafilm for 2 minutes
Degree of dry mouth is determined by visual analog scale (VAS)
Urine volume/void and frequency over 12 hours post dose is measured
Blood samples are taken for pharmacokinetics at pre-dose, and at, 1, 2, 4, 6, 10, 12 and 24 hours post dose
Food and water intake are standardized over the first 12 hour period

The study is a double blind, randomized, placebo-controlled, with sequences (5 treatments over 5 weeks) with the drugs being administered orally as a single dose. There is a one-week washout between treatments. The study population is chosen as follows:
Healthy volunteers
12 subjects
>18 years males or non-pregnant females
Weight 18-28 kg/m² BMI
No known allergy to antimuscarinic agents
No previous history of glaucoma, urinary retention, cardiac arrhythmias
No OTC medications, nutriceuticals or vitamins within 10 days of study enrollment and throughout the study

Assessments (except for urine output) is performed at: 0.5 hr and within 10 minutes pre-dose, 1, 2, 4, 8, 12, and 24 hours post dose. The following are assessed:
1) Stimulated salivary flow
2) Degree of dry mouth (VAS)
3) Urine volume/void over 12 hours post dose
4) Pharmacokinetics of tolterodine, active metabolite and pilocarpine

The standard safety precautions, such as physical exam, medical history, con-meds, ECG, hematology, clinical chemistry, urinalysis performed at screening and study termination, urine drug/alcohol screening at pre-dose for each period, vital signs (HR and BP) at: pre-dose, and at 2 hour intervals for 12 hours, and an awareness of adverse events throughout and between study period, are taken.

1. A pharmaceutical composition comprising a therapeutically effective amount of extended release tolterodine, or a pharmaceutically acceptable salt thereof, and a therapeutically effective amount of pilocarpine, or a pharmaceutically acceptable salt thereof
wherein, when the composition is administered to a patient, the peak plasma concentration for pilocarpine occurs at about 2.5 hours before the peak plasma concentration for tolterodine.

2. The composition of claim 1, wherein tolterodine, or a pharmaceutically acceptable salt thereof, and pilocarpine, or a pharmaceutically acceptable salt thereof, are together disposed in the same dosage form.

3. The composition of claim 1, wherein tolterodine, or a pharmaceutically acceptable salt thereof, is present in a dose of 4 mg.

4. The composition of claim 1, wherein pilocarpine, or a pharmaceutically acceptable salt thereof, is present in a dose of 5 mg.

5. The composition of claim 1, further comprising a pharmaceutically acceptable carrier, diluent, or excipient.

6. A method of treating a patient suffering from overactive bladder, the method comprising
identifying a patient in need thereof, and
administering to the patient a therapeutically effective amount of extended release tolterodine, or a pharmaceutically acceptable salt thereof, and a therapeutically effective amount of pilocarpine, or a pharmaceutically acceptable salt thereof;
wherein, after the administration to the patient, the peak plasma concentration for pilocarpine occurs at about 2.5 hours before the peak plasma concentration for tolterodine.

7. The method of claim 6, wherein tolterodine, or a pharmaceutically acceptable salt thereof, and pilocarpine, or a pharmaceutically acceptable salt thereof, are together disposed in the same dosage form.

8. The method of claim 6, wherein tolterodine, or a pharmaceutically acceptable salt thereof, and pilocarpine, or a pharmaceutically acceptable salt thereof, are administered separately.

9. The method of claim 6, wherein tolterodine, or a pharmaceutically acceptable salt thereof, is present in a dose of 4 mg.

10. The method of claim 6, wherein pilocarpine, or a pharmaceutically acceptable salt thereof, is present in a dose of 5 mg.

11. A method of alleviating a side effect of treatment for overactive bladder in a patient suffering therefrom, the method comprising
identifying a patient in need thereof, and
administering to the patient a therapeutically effective amount of extended release tolterodine, or a pharmaceutically acceptable salt thereof, and a therapeutically effective amount of pilocarpine, or a pharmaceutically acceptable salt thereof;
wherein, after the administration to the patient, the peak plasma concentration for pilocarpine occurs at about 2.5 hours before the peak plasma concentration for tolterodine.

12. The method of claim 11, wherein tolterodine, or a pharmaceutically acceptable salt thereof, and pilocarpine, or a pharmaceutically acceptable salt thereof, are together disposed in the same dosage form.

13. The method of claim 11, wherein tolterodine, or a pharmaceutically acceptable salt thereof, is present in a dose of 4 mg.

14. The method of claim 11, wherein pilocarpine, or a pharmaceutically acceptable salt thereof, is present in a dose of 5 mg.
